(19) United States
(12) Patent Application Publication
(10) Pub. No.: US 2026/0134973 A1
(43) Pub. Date: May 14, 2026

(54) METHOD AND SYSTEM THAT DELIVERS PERSONALIZED NUTRITION AND OVERSEES PATIENT PROGRESS TO IMPROVE BODY AND BRAIN HEALTH

(71) Applicant: Judith Gelernter, S. Setauket, NY (US)

(72) Inventor: Judith Gelernter, S. Setauket, NY (US)

(21) Appl. No.: 19/445,000

(22) Filed: Jan. 9, 2026

Related U.S. Application Data
(63) Continuation-in-part of application No. 19/335,880, filed on Sep. 22, 2025.
(60) Provisional application No. 63/697,629, filed on Sep. 23, 2024.

(51) Int. Cl. G16H 20/60; G16H 10/20; G16H 10/60; G16H 15/00; G16H 50/30; G16H 80/00

(52) U.S. Cl. CPC G16H 20/60; G16H 10/20; G16H 10/60; G16H 15/00; G16H 50/30; G16H 80/00

(57) ABSTRACT

The principle underlying this method is that the person's health improves when the microbiome within as well as the host is balanced and well-functioning. The method of this disclosure presents lifestyle choices that accommodate the superorganism of person plus microbiome. Examples of lifestyle choices are types of products safest to eat, use and wear, those safest to avoid. The system is simple to use in that it includes the minimum in the form of suggestion lists to reach better health, with links to references for those seeking scientific explanations. Users are asked from time to time to record their lifestyle choices and how their bodies responded. The logic of personalized suggestions derives from the health professional's knowledge and the scientific reference-supported Artificial Intelligence Analyst. The method and system depend upon continual internal and external cross-checks which enhances reliability.

### CROSS REFERENCE TO RELATED APPLICATION

This application claims the benefit of priority of U.S. provisional application number 63/697,629, filed 23 Sep. 2024, the contents of which are herein incorporated by reference. This application claims the benefit of priority of U.S. non-provisional application Ser. No. 19/335,880, filed 22 Sep. 2025, as a Continuation-in-Part thereof, the contents of which are herein incorporated by reference.

### BACKGROUND CHARACTERISTICS OF THE SUBJECT DISCLOSURE

The method of this disclosure presents choices for healthier foods and living spaces that have been demonstrated to support body and brain health. Potential system users are adults feeling fine who seek preventive measures against cancer and illness; adults who are caring for children with cognitive distress such as depression or autism, and adults experiencing cognitive distress and other chronic illnesses themselves. Symptom reduction will be the benefit for most with symptoms. Those without major symptoms might experience benefits such as more restful sleep, calmer mood, and higher energy.

Promise of improvement in body and brain health is based on the scientific understanding of a person as a superorganism (see National Institutes of Health Human Microbiome Project, 2007-2016) which requires nutrients both for the person and the microorganisms within. A fundamental principle is that the person feels and functions better with accommodation not only to the person but also to the person's microorganisms hosted within. Our lifestyle choices include types of products safest to eat, use and wear, for example, and types of products safest to avoid. It includes references to the literature for those seeking deeper understanding of the underlying science.

The system embodied in the subject disclosure is presented as short lifestyle choice lists that are practical for look-up, with nothing to remember, so that if a Patient choices and how his body responds to those choices, the choice list can be focused and personalized to him. The logic of personalization comes knowledge of discomforts and their potential causes from the health professional's training and experience, as supported by our Artificial Intelligence Analyst. The system depends upon continual internal and external cross-checks with verification among Patient, healthcare Provider, and the Artificial Intelligence Analyst to ensure that the Patient's wellness goals are being met reliably.

Method and logic of the actual system as disclosed here will continue to be refined as more users comment on the system, recognize its strengths and point out its flaws, which will be mitigated continually in effort to improve healthcare services.

Urgent need for our method and system. 21^{st} century eating and living conditions tend to favor some microorganism species over others—at times with the invasive species choking others and throwing the internal ecosystem out of balance—also known as gut dysbiosis or Small Intestinal Bacterial Overgrowth (SIBO). Dr Davis explains that “It is not uncommon for people with SIBO to suffer undiagnosed with various health conditions for years, even decades, enduring pain, disability, frustrating doctors'appointments that yield only partially effective or ineffective pharmaceutical solutions. Despite the flood of evidence, most practicing physicians remain unaware of this widespread and profound disruption of human health. Most conventional medical solutions fail to address the underlying cause: proliferation of unhealthy microbial species that export their inflammatory effects to other parts of the body” (Dr Davis, Super Gut, 2022) p. 75).

Significance of our method and system “Today healthcare workers spend most of their time caring for patients with chronic illnesses that last for years . . . . Eighty to ninety percent of clinic visits today are follow-up visits for [these] patients.” (Dr Naviaux, MD PhD, Foreword from Nathan, Toxic, 2018, p 6.). How to improve the situation? “We need an integrative approach whereby medical professionals treat the condition and functional medicine doctors, naturopaths and heath coaches help improve the terrain.” (Dr Nathan, Toxic, 2018, p.246) The present disclosure helps improve environmental conditions, or what Dr Nathan calls the terrain.

Role of environment vis à vis genetics. “The good news is that we can correct the influences that are altering our genetic expression and putting us at risk for chronic disease. That is the great lesson of the biological breakthrough represented in the mapping of the human genome . . . . that the translation process from genotype to phenotype is complex and dynamic as our genome responds to messages received from the environment. Therefore, to the extent that we can change the messages, we can also shape the response—and thereby affect our health outcomes . . . and in the explosion of research it set off.” (Bland, Disease Delusion, 2014, p.72). Specifically with respect to food, wrote Dr Hyman, “Food contains information that talks to your genes, turning them on or off and affecting their function . . . Food is the fastest-acting medication you can take to change your life. This discovery is called “nutrigenomics.” (Hyman Ultra Mind Solution, 2009, p. 37).

Subject disclosure aims to lessen inflammation. “The underlying theme for all of these conditions, whether they are triggered by toxins or infectious agents, is inflammation.” (Dr Nathan, Toxic, 2018, p.39) The stimulus might no longer be present, but the chronic illness is maintained through a persistent inflammatory response. (Dr Nathan, Toxic, 2018, p.127) Our method of on-going feedback and nutrient suggestions aims to lessen that inflammatory response.

Subject disclosure follows a systems biology approach to inflammation. “The gut is the starting point of an awful lot of chronic illnesses that seem to have nothing to do with the stomach; it's the launch pad for numerous symptoms affecting parts of the body far distant from the gastrointestinal area.” (Dr Bland, Disease Delusion, 2014, p. 102). The 21st century understanding of how human body systems work and the importance of a balanced gut ecosystem has led functional medicine pioneers to devise the 4R protocol to improve gut dysbiosis. “The Four R program represents a clinically proven approach to managing complex health problems associated with imbalances of the assimilation-elimination process.” Bland continues “co-equal in importance is the process of detoxification when what is ingested is not properly assimilated or eliminated.” (Bland, Disease Delusion, 2014, p. 118). Our approach essentially presents the patient with recommendations for what the Health Coach believes is needed by the individual and also what could be removed for optimal body functioning.

Subject disclosure effectiveness. Our method rests on the proven, published approach of dieticians and doctors such as Dr Bland (Disease Delusion, 2009), Dr Kellman (Microbiome Diet, 2014), and Dr Davis, (Super Gut, 2022), and Dr Hyman, (UltraMind Solution, 2009). Our system to deliver personalized recommendations is enhanced by multiple counter-checks, whereby questions are asked the user at different times each month, and the user's answers are assessed by the Artificial Intelligence Analyst as well as by potentially more than one trained Health Coach.

System personalized to you. Dr Bland, pioneer of functional medicine, wrote “We need a new model of care that will personalize treatment to the individual's particular genetic makeup, environment, lifestyle, and diet.” (Dr Bland, Disease Delusion, 2014, p. 13). Moreover, it has been acknowledged that different foods are optimal for different people (Segal and Elinav, Personalized Diet, 2017). The feedback loop of our system allows personalization by asking questions periodically of the patient an on-going way to determine whether the nutrient suggestions are making the patient feel better, and whether the program as a whole is meeting the goal of improving the patient's well-being.

Method and system aim for cognitive as well as physical improvement. Activated immune defenses have been discovered experimentally to cause foul mood and sickness behavior (Dr Lyman, Immune Mind, 2024, pp.35-48). So not surprisingly, the nutritional methods that lessen inflammation have been demonstrated in clinical practice to provide cognitive improvement (for example, Dr Campbell Mc-Bride, Gut and Psychology Protocol, 2009; Hyman UltraMind Solution, 2009; Kellman Whole Brain, 2018). Inflammation has been associated with thyroid function, and thyroid dysfunction can be as a major influence on brain chemistry and potential disorder (R. Arem, Thyroid Solution, 2007, p82). Retainment of the primitive reflexes due to inflammation in the very young also, can cause cognitive difficulties. We recommend that some will benefit from Dr Melillo's neurosensory exercises (see for example, Leisman and Melillo, Evaluating Primitive Reflexes in Early Childhood, Journal of Paediatrics and Child Health, 2025).

Access to cognitive services. A treatment gap has been recognized between the proportion of the population in need of cognitive services, and the proportion that actually receive that help. Even if seen by a therapist, Dr Kazdin reported, 50% at best do recover. (Dr Kazdin, Behavior Research and Therapy, 88(2017 ), 7-18.) What to do? Doctors Kaplan and Rucklidge proposed that a healthier diet could influence the treatment gap. “What we really need are treatments that are easy to follow and affordable - that maximize reach . . . so we can help more people” (Dr Kaplin and Dr Rucklidge, The Better Brain, 2021, p114, 115). The method of the subject disclosure can help narrow that treatment gap by offering easy access to many who need it.

System practicality. Our method derives from the inventor's implementation and refinement of the method on herself as published in gut-health books described above as well as scientific research articles. But how many people today want to read a several-hundred-page book or study the research? Most of us prefer not to study tax law in order to do our taxes responsibly each year, or learn auto mechanics to order to ensure our cars drive safely and reliably. Most want to learn the minimum to perform the maximum—with learning more optional. The method of this disclosure is presented as choose-it-yourself lists that require only a few minutes of data entry each month. Learning is optional.

System assistance in changing habits. We found during our Product-Market Fit qualitative research supported by the National Science Foundation in autumn 2024 that of those parents who attempted nutritional methods for children, many parents need psychological assistance in transitioning the child to different foods. Occasional visits to doctor or dietician did not, for our representative sample, supply the help that was necessary to transition to, or continue to eat, the foods recommended. That is why our service includes the help of a Food Therapist at the beginning period of adjustment.

System continued encouragement. Immune conditions raise endotoxins which lower mood and raise anxiety, driving the patient away from doing things. (Dr Lyman, Immune Mind, p. 38). Consequently, some might not feel like participating in a wellness protocol. This is confirmed by Dr Hyman, who confessed: “[M]y challenge [being a doctor] is always to help people connect to meaning . . . . The development of . . . a sense of connection, belonging, and contribution must be nurtured and fed.” (Dr Hyman, UltraMind Solution, 2009, p. 283) Our response to this situation was to add warmth and connection. Optional services for users include a social media-type platform in which participants are encouraged to have their say and interact with others in the program community on relevant topics. Another optional service is our Buddy system, with an anonymous match to another participant or a caregiver in a similar stage of the program. Thus, we took the advice of Dr Moday who wrote that “Doing [an] immune restoration plan with a friend can help you stay motivated and make it a whole lot more fun.” (Moday, Immunotype Breakthrough, 2021, p.225).

Benefits, risks and risk mitigation strategies. Benefits of raising the nutritional content of meals and lowering potential toxins in additives and household products should be seen in lessening physical and cognitive distress and increasing quality of life. Risks will come from the additional time preparing food with sharp equipment. Peelers, graters and sharp knives might slip and cut anyone rushing through vegetable preparation. The ideal of more expensive, organic ingredients makes it unlikely that the base diet will be available pre-made, however, many organic vegetables can be purchased pre-cut, pre-sliced or frozen. These are faster and safer to prepare and retain most of their nutritional value.

Selfcare might be medicine's future. In the words of Dr Bauer, Mayo Clinic guide to Integrative Medicine, 2017, p.9 “People who take an active role in their health care experience better health and improved healing. It's a commonsense concept that's been gaining scientific support for several years now.”

A thorough review of the prior art of gut healing demonstrates many approaches to nutrition and selfcare that include elements of the method, practicality, access, motivation and so on, but nothing that combines them into a single system with enhanced internal reliability for personalized nutrition.

Our method depends on some aspects of the prior art:
a) Particular nutrients extracted and encouraged for greater health. For example, consider the compound found in some mushrooms called psilocybin, to be produced as a dietary supplement (Londesbrough et al, 2025, U.S. Pat. No. 12,377,112 B2).
b) Set of nutrients in recommendation for foods. For example,
   A healing diet with attention to immune system support that requires restriction of eating times, and restriction of calories from carbohydrates—which has been patented to treat depression and Alzheimer's for example (see Longo, 2024, U.S. Pat. No. 12,029,231 B2 for depression), (see Longo et al, 2025, for Alzheimer's US 2025/0098723 A1.
   A similar set of foods was proposed also by Longo for the purpose of health span extension (Longo, 2025 US 2025/0177466 A1). Note that a restricted eating time window and carbohydrate restriction is one small part of our invention.
c) Software that recommends what to eat based on individual data:
   Apps for weight loss (Noom in noom.com, Lifesum in lifesum.com, HealthyEating healthyeating.com), nutrition during pregnancy (Ovia Pregnancy Tracker), or personalized for your chronic condition (Heali app) or user feedback based on body physique or other goals (LeBlanc-Bazinet et al, 2023 U.S. Pat. No. 11,581,085 B1),
   Apps that generate recommendations based on the individual's microbiome data (Grady, 2024, US 20240412836 A1), or (Angelides, 2022 U.S. Pat. No. 11,315,686 B2), or the Viome company (Viome.com) or apps that are software as a service such as Revero (revero.com),
   Apps that generate nutritional recommendations based on biometric data, sensor data or lab data regarding the individual's metabolic system (Neumann, 2023, U.S. Pat. No. 11,688,507 B2), or by means of sensing devices or biometric data that takes measurements (Vleugels et al 2025, U.S. Pat. No. 12,367,962 B2), or metabolomics data (Kaddurah-Daouk et al, 2025, US 20250062037 A1).
   Apps that generate recommendations for meals based on a food nutrition score (Patent CN 105260594A, Individual nutrition evaluation system, 2016); or instruct a person based on food data such as the Nutrino company (Nutrinohealth.com).
   Apps that provide communication channels for recommendations with a health coach (Wellfedwellness)
d) Artificial intelligence approaches that consider data from the individual or a group with similar characteristics to offer recommendations (Kirk D, Catal C, Tekinerdogan B. Precision nutrition: A systematic literature review. Computers in Biology and Medicine. 2021 June; 133:104365.)
e) Physical treatment approaches rather than recommendations. A physician performs the Fecal Microbiota Transplant by transplanting microbiota sample from a well person into the colon or possibly small intestine of the person with gut dysbiosis. Patents cover this domain (Adams, 2022, US 2022/0088089 A1). Note that results are mixed over the long term. Advanced methods of this nature explore transplanting entire microbial communities or filtering particular items from feces. (Yu Y, Wang W, Zhang F. The Next Generation Fecal Microbiota Transplantation: To Transplant Bacteria or Virome. Adv Sci (Weinh). 2023 December; 10(35):e2301097.)
f) Brain wellness software
   There are a good many mental health apps that do not consider the gut. For example, there are some that work to relieve anxiety (such as the Mindshift or the Happify app), or support meditation to aid relaxation (such as the Calm, or Headspace apps), or offer a talk-it-out strategy (such as the Talkspace app).
g) Brain wellness neurosensory program. Dr Melillo's Brain Balance program aims to strengthen the side of the brain weakened by inflammation and help create more connections between sides of the brain. (Dr Melillo, Reconnected Kids, 2011), (Dr Melillo Disconnected Kids, 3^{rd} edition, 2024)
h) The nutritionist food diary approach
   Which are really the right foods for each individual? Short of diagnostic tests regarding food intolerances, there is the dieticians'method of food journalling, also known as the food diary. The purpose is to remove from the diet those foods that create symptoms for the patient, and therefore most likely, inflammation. The patient records foods, supplements and time eaten—and the symptoms that ensue (See for example, Jacob, Digestive Health, 2013, p.154.).
i) Physician microbiome evidence-based approaches. (See for example, Bland, Disease Delusion, 2014; Kellman, Microbiome Diet 2014; Hyman, UltraMind, 2008; Davis, Super Gut, 2022.) Basically this treatment model incorporates a phased approach to re-balance the gut microbiome. On an easily-digestible diet, foods that many micros find damaging, known as antimicrobials, are given to remove noxious bacteria, along with nutrients to heal gut damage. The final focus is to restore beneficial bacteria by ingesting probiotics and fermented foods that

contain probiotics. The protocol to heal the gut is complicated by the fact that it requires multiple types of food, taken at particular times of day (esp. on empty stomach in the morning), and over different phases of the individual's healing.

As can be seen, there is a need for an effective method and system that delivers personalized nutrition and other information and oversees patient progress for improving body and brain wellness.

### SUMMARY OF THE SUBJECT DISCLOSURE

Patient choice and personalization. We aim help patients feel better by offering suggestions toward healthier foods and living spaces among which patients can choose. We empower patients with ability to choose, and to learn more about any suggestion, rather than delivering instructions. We ask patients periodically what they choose and how they are feeling, so that the Health Coach can offer insight into what can be suggested to help the patient at this stage. Thus, the personalization method functions by taking information from the patient continually in a sort of feedback loop.

Reliability of personalized suggestions is enhanced by continual checks and balances among Patient, Provider and Artificial Intelligence Analyst. The Healthcare Provider will have the final decision, while the Artificial Intelligence Analyst acts like a research team offering an independent to the Healthcare. For example, the Patient verifies that that System is effective in its healing goal by reporting general health measures that reflect the quieting of the immune system, such as level of energy and mood. The Artificial Intelligence Analyst alerts the Healthcare Provider should the general measures or specific discomforts remain stable over too many consecutive months.

Effectiveness from current research in different healthcare domains. The present disclosure to improve body and brain health is the only digital health span as a service that unites information in its Knowledge Base from scientists and physicians who are authorities in different if complementary domains, on gut healing, personalized nutrition, detoxification and inflammation, endocrinology, neurology, and others with continued oversight from a professional Health Coach. Key to method effectiveness is to keep abreast of on-going scientific research and personalize suggestions based on the individual's responses and reactions.

Practicality. It is an at-home system for which practicality is key. The massive information is made manageable by attempting to present what the patient should know when he needs to know it. On-screen overviews are available even when using a mobile phone to following a grocery list in the supermarket. An affordable subscription is open to anyone with Internet access. Data and communications are protected within secure, encrypted channels mandated government regulations. Real time, encrypted video or voice meetings between Food Therapist and Patient, or professional Health Coach and Patient are optional.

Long-term healing. The aim is for health to improve greatly—but to get there, some patients will need to continue for a long time. To inspire and motivate our patients to continue, the system includes a memory of how the patient used to feel to show them their progress (visualization of progress); a Buddy system in which—at their choosing—we pair two patients or two caregivers to give each other emotional support along the journey; and a whiteboard to use as an internal social media platform in which patients can have their say on a healing-related topic or comment on each other's comments. In this way, we hope many will stay through the stages of healing, and that patterns set during the program will become habit and make the system unnecessary long-term.

These and other features, aspects and advantages of the present subject disclosure will become better understood with reference to the following drawings, description details and claims.

### BRIEF DESCRIPTION OF THE DRAWINGS

FIG. 1 is a diagrammatic view of an exemplary embodiment of the subject disclosure, illustrating different services available from Patient, Provider and Administrator Portal Interfaces.

FIG. 2 is a schematic view of an exemplary embodiment of the subject disclosure, illustrating a portion of the interface of the microservice called Intake.

FIG. 3 is a schematic view of an exemplary embodiment of the subject disclosure, illustrating a portion of list for Foods.

FIG. 4 is a schematic view of an exemplary embodiment of the subject disclosure, illustrating a portion of the interface from the microservice called Mealtimes.

FIG. 5 is a schematic view of an exemplary embodiment of the subject disclosure, illustrating a portion of the interface from the Symptoms microservice.

FIG. 6 is a schematic view of an exemplary embodiment of the subject disclosure, illustrating a portion of the interface from the microservice called Nonfoods.

FIG. 7 is a schematic view of an exemplary embodiment of the subject disclosure, illustrating the card-like interface of MyPlan.

FIG. 8 is a schematic view of an exemplary embodiment of the subject disclosure, illustrating the card-like Patient Response interface, where what is being responded to is the plan proposed on the other side of the flip card.

FIG. 9 is a schematic view of an exemplary embodiment of the subject disclosure, illustrating a portion of the microservice called Health Tracker.

FIG. 10 is a view of an exemplary embodiment of the subject disclosure, illustrating a potential graph that could be compiled from Health Tracker data.

FIG. 11 is a schematic view of an exemplary embodiment of the subject disclosure, illustrating the data display for the optional InfoShare platform.

FIG. 12 is a diagrammatical view of an exemplary embodiment of the subject disclosure illustrating potential contents for the Knowledge Base module.

FIG. 13 is a diagram that shows an exemplary embodiment of the subject disclosure with respect to how we arrive at the patient's starting foods.

FIG. 14 is a view of an exemplary embodiment of the subject disclosure, illustrating features that combine microservices for, inter alia, improving antimicrobial effectiveness.

FIG. 15 is a chart of an exemplary embodiment of the subject disclosure, illustrating services that may be involved in how we personalize nutrition.

FIG. 16 is a schematic view of an exemplary embodiment of the subject disclosure illustrating health span as a service, showing potential patient interaction with the system and the method's effects on a patient over a period of time

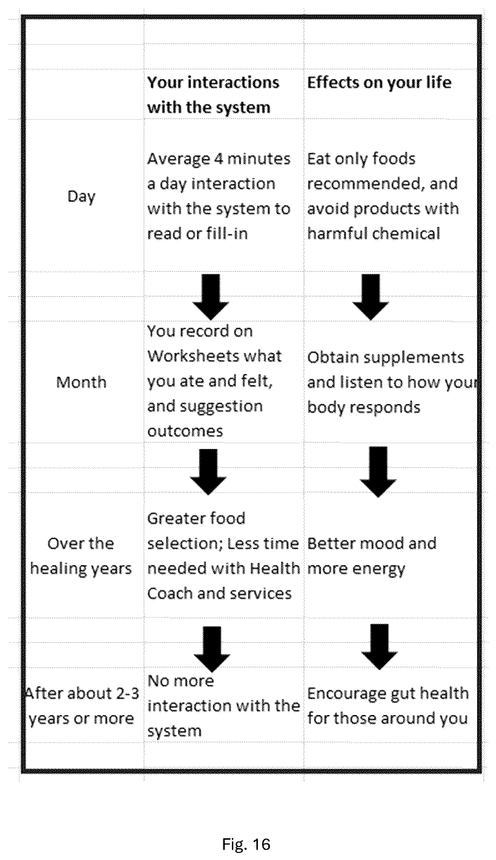

### DETAILED DESCRIPTION OF THE SUBJECT DISCLOSURE

### I. Overview

In one aspect of the present subject disclosure that is a computer-implemented method for updating healthcare suggestions for a patient, the method includes the following: receiving, via a profile interface of an intake user interface module executing on at least one processor, a plurality of profile inputs from the patient; presenting, via a food data user interface module executing on the at least one processor, food data comprising a plurality of food items, each food item having an associated consumption indicator; presenting, via a meal timing module executing on the at least one processor, recommended meal times and micronutrient recommendations; presenting, via a toxin information module executing on the at least one processor, information regarding potential toxins in household products; generating, using an artificial intelligence system executing on the at least one processor, treatment suggestions based on the plurality of profile inputs and indicators from the food data user interface module, the meal timing module, and the toxin information module; transmitting the treatment suggestions to a healthcare professional for review receiving, from the healthcare professional, approved treatment suggestions for transmission to the patient; transmitting the approved treatment suggestions to the patient; receiving, via the user interface, patient feedback regarding the approved treatment suggestions; and updating at least one of the food data user interface module or micronutrient recommendations based on the patient feedback.

In another aspect of the present subject disclosure, the computer-implemented method for updating healthcare suggestions for a patient further includes the following: periodically updating the treatment suggestions based on patient feedback received in response to previously transmitted treatment suggestions; correlating macronutrients and micronutrients consumed by the patient with patient-reported symptoms over time; and modifying food recommendations by reducing or removing foods associated with the patient-reported symptoms; retrieving, by the artificial intelligence system, treatment information from clinical or scientific research publications; and presenting the treatment information to the healthcare professional for selection of the approved treatment suggestions; providing, via an empathy module executing on the at least one processor, a communication interface enabling the patient to exchange empathy-related communications with other patients; and providing, via a community module executing on the at least one processor, a community interface enabling the patient to interact with a wellness community, wherein educational content presented to the patient is formatted in a same manner as the user interface for receiving the patient feedback; personalizing a task interface to display only tasks relevant to a current treatment stage of the patient, wherein receiving the patient feedback includes receiving a description of a discomfort experienced by the patient; receiving a location indicator to show where the discomfort is experienced; receiving a severity indicator indicating a severity of the discomfort; and comparing the severity indicator to a previously reported severity indicator from a prior feedback cycle; periodically updating macronutrient recommendations and micronutrient recommendations throughout a treatment course based on the patient feedback, wherein presenting the recommended meal times includes: determining an optimal time for presenting the approved treatment suggestions to the patient based on the patient's treatment stage; and indicating for the approved treatment suggestions the optimal time of day. Presenting information regarding potential toxins comprises: providing information regarding safer alternatives to the potential toxins; and gradually presenting the safer alternatives over a period of time; receiving, via the user interface, a plurality of health goals from the patient; tracking progress toward the plurality of health goals; and displaying a graphical visualization of the progress toward the plurality of health goals; collecting anonymous statistical data regarding patient responses; analyzing the anonymous statistical data to generate metrics comprising at least one of: patient healing rate, user interface usability, or healthcare professional effectiveness; and modifying the method based on the metrics.

In yet another aspect of the present subject disclosure, a method to update a patient's healthcare suggestions based on his response to questions, the method executed in digital form by at least one processor, includes the following: prompting a patient for a profile through a profile interface represented via an intake user interface and module, wherein the profile comprises a plurality of profile inputs; representing food data with a user interface, by way of a module, for each food datum a consumption indicator is provided; representing when meals should be eaten for maximum benefit, and what micronutrients would enhance health by way of a module; representing potential toxins in common household products via the user interface by way of a module; identifying treatment suggestions independently by Artificial Intelligence methods, and by a Healthcare Professional upon receipt of module indicators, wherein the treatment course is based on the plurality of profile inputs and module indicators; sending treatment suggestions to the patient by the decision of the Healthcare Professional, wherein the Patient gives feedback about whatever treatment suggestions were tried, via the user interface; and upon receiving that patient feedback input via the user interface, updating the modules as for foods or dietary supplements based in part on that patient feedback, wherein the identified treatment suggestions are periodically updated upon receipt of a new response to a new treatment suggestion and in light of patient progress, whereby macro- and micro-nutrients are calibrated by time with patient symptoms in a sort of “food diary” or “food journal” approach, so that foods likely to be associated with symptoms can be either lessened in amount or removed from the patient's personal food list temporarily. Treatment suggestions might derive from empirical knowledge of the Healthcare Professional, or from up-to-date clinical or scientific research publications retrieved by Artificial Intelligence means, and for the provider to choose which treatments to send to the patient. The system includes options of an empathy module and a community module so that the patient has the option of giving or receiving empathy over the course of healing, and can feel connected to this selfcare community.

In still yet another aspect of the present subject disclosure, a system for the above-mentioned method includes interfaces in which the “feedback” data from different modules is presented in the same way in order to improve consistency and ease-of-use, in which not only the program, but also the task interface is personalized, so that only the tasks the patient should focus on at present are available so as not to clutter the screen or overwhelm the person. And in which the patient is offered the opportunity at every cycle not only to report to the provider what is the discomfort, but also where is the discomfort and to what severity it is compared to what was reported in the cycle prior, in which the system has the ability to update nutrition—both macro and micronutrients—periodically over the entire course of healing based at all times on the patient response (in a feedback loop, for example). And in which the patient is presented with suggestions when throughout the day it might benefit that patient most, and with suggestions for his stage at the right time in the healing program based on his own responses, and in which potentially harmful environmental toxins are shown in products where they might be found, and are accompanied by safer alternatives; and each toxin is delivered gradually over time so as to make change practical. And in which the patient has the opportunity to track several health goals, and see those goals visualized in a graph. And in which the system provides metrics to evaluate itself by taking anonymous statistics on treatment effectiveness, for example, in terms of interface usability, or in terms of provider's success rate.

### II. EXAMPLES

The following detailed description represents one way of making and using exemplary embodiments of the subject disclosure. The description is not to be taken in a limiting sense but is made merely for the purpose of illustrating the general principles of the subject disclosure, since the scope of the subject disclosure is best defined by the appended claims.

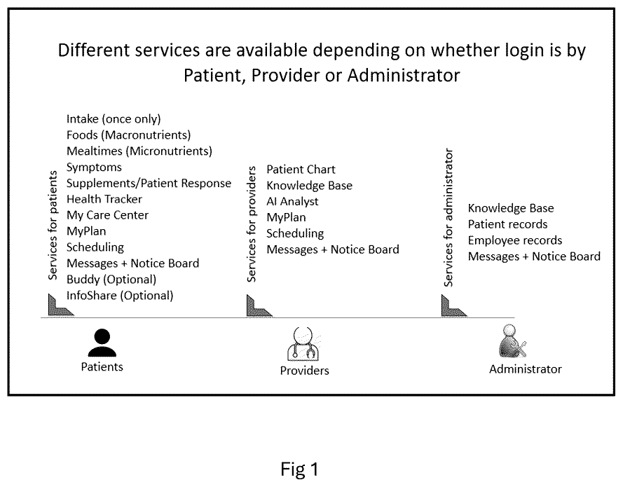

The practical method is delivered by software that is divided into services. Patient services are delivered in a Patient Portal, Provider Services are delivered in a Provider Portal, and Administrator Services are delivered through an Administrator Portal, each of which is accessed through different login credentials. Services relevant to each portal appear in FIG. 1. Each Portal includes its own metrics for value-based care because the goals of the patient, those of the Health Coaches, and those of the Administration itself differ in detail.

Our aim is the patient's wellbeing. The Services in the Patient Portal for patients are delivered in such a way as to make them particularly easy-to-follow. The interface shows only what is needed at the right time—nothing more. Consistency that encourages ease of use comes from the same lists being transformed into worksheets with data-entry fill-in circles called radio buttons. This is so that the Health Coach with the Artificial Intelligence Analyst support can review patient nutritional choices and make suggestions. Consistency appears also in describing symptoms not only in time and location but also in intensity using dials which appear in several of the patient services.

The Mealtimes and Foods mainly focus on nutrition. The Foods section lists easily-digestible foods for the patient to enjoy, while Mealtimes reminds the patient to take food supplements, for example, when the patient cannot for some reason tolerate the quantity of the food required per nutritional need. Mealtimes also encourages body repair by reminding the patient not to eat during the nighttime and potentially delaying breakfast. Why? This is the autophagy that supports DNA repair and lowers inflammation. (Mattson MP, Longo VD, Harvie M. Impact of intermittent fasting on health and disease processes. Ageing Res Rev. 2017 October;39:46-58.)

The Plan cards help personalize nutrition. The cards contain suggestions personalized to each individual patient. Mealtimes and foods lists update only if a positive response has been received from that individual. New suggestions are sent throughout the program as the patient's health improves.

NonFoods alerts the patient to potential toxins in even minute quantities in products that, with continued use, can threaten health. A different potential toxin, product categories that might contain that toxin, and safer alternatives are introduced periodically.

Supports for the Patient include motivation that should derive from viewing own's own health progress chart; emotional support in a Buddy System so that you can talk openly to one person also in the program, and the support of the community within the program in our social media platform.

### III. Glossary

The sole purpose of the glossary definitions is to make the specification more understandable to readers, rather than as a novel addition to the literature.

### Technical and Business Glossary

Closed-loop system=a system that takes feedback (in this case, user input data) and adjusts output continually according to that feedback

Digital selfcare=collection of procedures needed to allow patient or next of kin to take care of patient's health. Usually procedures are overseen by a specialist on the basis of documentation collected on the digital platform

### Knowledge Base=Indexed Table of Underlying Assumptions About Nutrition and other aides

Value-based care=“value” to patients is improved quality of life, “value” to payers is reducing total costs of care, “value” to providers is improving outcomes while being paid fairly for cases of increasing complexity. (Gonsiorek, McKnights'Long-term Care News, Jun. 4, 2025).

### Biology Glossary

Autophagy=a type of recycling that produces new building blocks and energy for cellular renovation and homeostasis (Mizushima N, Komatsu M. Autophagy: renovation of cells and tissues. Cell. 2011 Nov. 11; 147(4):728-41.)

Biofilm=a film created by usually different species of microorganisms so that they can adhere to a surface and use each other as protection and to increase survival

Diet=the set of foods people eat every day. Note that diet does not include the idea of what the body needs, nor does it include the idea that substances on the skin's surface for example become part of the body. Diet is not the subject of this invention; the subject is nutrition.

Dietary supplement=vitamins, minerals or bioactives taken in small amounts that supplement macronutrients, or foods.

Gut dysbiosi=An imbalance in between beneficial, commensal and pathogenic microorganisms in the gut. This tends impede the immune system, and impede health. (Reference: Van Hul M, Cani P D, Petitfils C, De Vos W M, Tilg H, El-Omar E M. What defines a healthy gut microbiome? Gut. 2024 Oct. 7;73(11):1893-1908.) Also see leaky gut, and also see SIBO in this Glossary.

Health=good health is freedom from disease owing to strong immunity, and the gut is the epicenter of immunity (Moday, The immunotype breakthrough, 2021, p.125).

Also see Wellness in this Glossary

Healthspan as a Service (HaaS) digital tool in which patients receive suggestions to improve their quality of life. Healthspan has been linked to gut microbiome balance health (see patent Johnson et al, 2024 U.S. Pat. No. 12,080,432-B1)

“Herx” or Herxheimer response Named for physician Herxheimer who first described it, the symptoms of the reaction (such as fever or fatigue) indicate a strong immune response

Immune system the English “immune” derives from the Latin meaning ‘free from’ as free from a burden, duty, or in the case of the human body, free from disease. Immune defenses cover the entire body, including the brain.

Inflammation is an immune system reaction that is triggered when your body gets injured or infected to protect and heal. It can cause redness and swelling at the site of an injury and mucus when we get a cold, and it can cause foul mood and sickness behavior. (Dr Lyman, Immune Mind, 2024, “Sick Sense” section). However, an inflammatory response that is triggered too much might produce inflammation more serious than the original illness or injury. (Dr Moday, Immunotype Breakthrough, 2021, section called “Double-Edged Sword of Inflammation”)

Leaky Gut which is an imbalanced collection of microflora, either impede healthy gut mucus or worse, adhere in clumps to the gut walls. The result is that the walls become porous and leak toxins into the body that would otherwise remain in the gut and be excreted. Also see gut dysbiosis, and also see SIBO in this glossary

Microbiome is a biome—a set of living microorganisms throughout our digestive tracts and on our skin biomes. These microscopic organisms account for about half of the cells in our body. They are microbes and historically they have been largely ignored as a factor in human health. Valcourt, Systematic, 2017, p.190

Non-pharmacological: Our method in standard applications does not involve medications or pharmaceuticals.

Nutrition=nourishing substances taken into the body to sustain life, as though eating, breathing, and skin absorption. Nutrition is not the same as Diet; see Diet entry in this Glossary

Personalized nutrition=precision nutrition=individual nutrition science has demonstrated that different people require different levels of nutrients (S, Campos-Medina L, Leal-Mercado L. Personalized nutrition: the end of the one-diet-fits-all era. Front Nutrition. 2024May 24;11:1370595.) Artificial intelligence has been used to personalize nutrition (Kirk D, Catal C, Tekinerdogan B. Precision nutrition: A systematic literature review. Computational Biology and Medicine. 2021 June;133:104365.).

Probiotics Beneficial microorganisms that help us, their human hosts, help digest our food and get rid of some microorganisms that are pathogenic

SIBO Small Intestinal Bacterial Overgrowth. Multiple species of microorganisms inhabit the colon with some in the small intestine. When conditions favor some species over others, some species might proliferate to disturb the balance—this the overgrowth. Also see gut dysbiosis in this Glossary, and also see leaky gut.

Superorganism=the person and also the microbiome that person hosts

Systems biology is an interdisciplinary approach to try to understand how parts of biological systems include microorganisms work together, as compared to medical training which divides into human body systems and tends to focus on one system exclusively, such as cardiology, gastroenterology, and so on.

Wellness=the state of good health, that is, being well. Also see Health in this Glossary

### IV. Description of Some Aspects of Our Method That Make it Effective

Restoring health with respect to digestive and endocrine disruptors. “. . . [A] growing community of physicians agree . . . that these phenomena [that cause chronic illness, for example] are triggered by a significant increase in the toxicity of the world in which we live. We are exposed to thousands of chemicals that simply did not exist fifty years ago. We are also exposed to massive amounts of electromagnetic fields that did not exist fifty years ago; worse, we hold them in our hands, to our ears, and on our bodies all day long.” (Dr Nathan, Toxic, 2018, pp.32-33). This is the purpose of our NonFoods microservice.

Restoring health with the respect to immune response. In addition to removing potential hazardous substances in NonFoods, we offer Mealtimes that incorporates the idea of delaying breakfast to increase the capacity of our cells to self-repair.

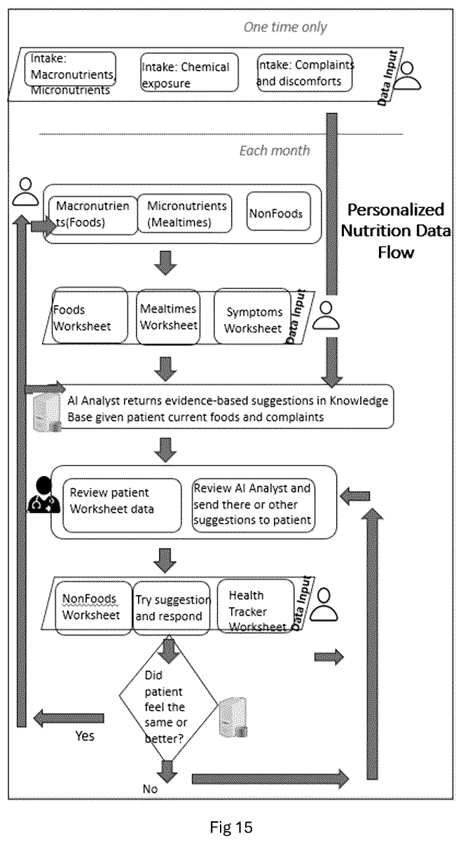

Restoring health with respect to nutrition.—Translates into our Foods recommendations. Dr Li emphasizes the importance of food as medicine in his Eat to Beat Disease, 2019; Dr Gundry includes food lists in his Gut Brain Paradox, 2025, as does Dr Hyman in “What the heck should I eat?” The problem is, however, that foods are individualized. That is why our system considers individual factors to recommend what foods should be eaten, and in some cases, in what amounts. This uses many components of our system, as shown in FIG. 15.

Restoring health with respect to mood. Simply put, inflammation reduces ‘feel good’ neurotransmitters serotonin and dopamine. That is why many people who suffer chronic illness deal with anxiety or depression. (Lyman, Immune Mind, 2024). Our method relieves inflammation, for example, by suggesting to patients which foods and environmental toxins to remove as the gut heals and as inflammation subsides. Nutrients can aid gut repair. And certain probiotics can add another level of calm. (Puri S, Shaheen M, Grover B. Nutrition and cognitive health: A life course approach. Front Public Health. 2023 Mar. 27;11:1023907.) Calmer mood is an expected outcome for all those who stay with the program through the final phase of adding fermented foods and probiotics. Restoring health with respect to brain imbalance. Inflammation in children has the potential to delay growth of connections between the sides of the brain. This leads to a dominance of one side of the brain over the other, with accompanying behaviors that might be extreme—such as acute anxiety (with dominance of the right hemisphere), or aggression (with dominance of the left hemisphere). Research reveals that integrating primitive reflexes can aid brain development, and motor and neurosensory exercises have the capacity to strengthen the weaker side and increase connections between the sides (Melillo and Leisman, Neurobehavioral Disorders of Childhood, 2009). This is borne out by the success of Dr Melillo's Brain Balance method, and we refer some patients to the Brain Balance exercises.

Continual motivation in seeing progress, feeling emotional connection, and participation in this selfcare community are in-program services for patients. These services are optional.

### V. Descriptive Examples of How our Method Differs From Others

(1) Combination of multiple healthcare protocols toward gut healing and restoration of strength after chronic inflammation that not elsewhere combined. For example, our method includes dieticians'food diary approach, the 4R (Remove, Replace, Reinoculate and Repair), now called 5R (with Rebalance) approach of functional medicine, nutrition personalization, a methodological system for the trace-chemical removal, and suggestions to lessen the inflammatory response. This is within an easy-to-use practical system with professional Health Coach oversight throughout the program.
(2) Internal and external checks and balances by Patient, Health Coaches and Artificial Intelligence Analyst add reliability to the personalized suggestions. The Patient continually tells us not only how he feels, but whether the Health Coach suggestions he tried were effective. The AI Analyst reviews the Patient's symptoms and monitors his progress, as well as taking anonymous data on each suggestion's relative effectiveness. The Health Coach reviews the AI Analyst suggestions and has the final decision which to send to the Patient. The Health Coach as well as the Administrator hold the ability to update the Knowledge Base on which the AI Analyst depends.
(3) Personalized foods and dietary supplements are patient choice. Additional information for a deeper understanding of the suggestions empower the patient to make wise decisions.
(4) Some patients or caregivers will be busy and will choose not to learn more. For these, the system accommodates by making appear on screen only what is needed.
(5) Video visits between Patient and Providers in most cases are optional. Instead, the Provider reviews the Patient's current condition and progress at regular intervals throughout the Patient's time with the selfcare program. Strongly encouraged at the beginning of the program for parents caring for a child is a meeting with the Food Therapist due to the difficulty of changing a child's eating patterns.
(6) Patients have the opportunity to discover some of “root causes” of their discomfort themselves. While detective insights from Health Coaches and our AI System can be quite useful, the Foods and NonFoods sections together might cover some elements that could jeopardize health.
(7) Our system incorporates a behind-the-scenes referral system among Health Coaches so that if one Health Coach is unsure how to help a particular patient, he can send that patient's chart to another Coach along with a question. In this way, no professional is embarrassed or forced to pretend to be an authority on everything, and the patient reaps the benefits.
(8) Our system offers outlets of inspiration to keep Patients motivated to continue. Empathy takes the form of an in-portal matched buddy, who is another user of the system, with whom the patient or caregiver might choose to share experiences about his healing journey over a channel that is encrypted. Connectedness to the community takes the form of a platform to post thoughts or add to others'thoughts on topics relevant to the program.
(9) Our system gives extra attention to those patients who might not be improving steadily, thanks to the Artificial Intelligence Analyst monitoring of the patient's Health Tracker data. An alert will be sent to the Health Coach for the long-term sort of patient information that the Health Coach focusing at the patient's current complaints may or may not notice.

### VI. Descriptive Examples of How our System Is Designed for Patient Ease of Use

User Interface to Improve User Experience
   Microservice data entry in many ways is consistent. For example, almost the same Foods and Mealtimes worksheets serve for read-only or data entry, and the same dials (for intensity/severity) are used for the Symptoms and Health Tracker.
Data entry requires appear only at the time needed
   For example, the System presents the Health Tracker microservice to each Patient only when it is time to enter Health Tracker data. As another example, so as not to overwhelm the patient or family, only a single Nonfoods chemical appears at a time
Minimum to write
   For example, many data entry Worksheets include choice selections. Writing sentences is optional.
Minimum to learn
   Recommendations are straightforward lists, with references throughout for those seeking more information to make a decision.
Empowering to choose
   Patients may choose to follow suggestions or to look-up references first and learn more before making their own decisions.

### VII. Descriptive Examples of How Our System is Designed for Healthcare Provider Ease of Use

Continuity of care. A provider already familiar with a patient's background is likely to be the provider to take any scheduled video meetings. Another Provider might help, but that would take more time to get acquainted with the patient's background.
Prioritization as well as aggregation of information for each patient, each time the Health Coach reviews the information
Artificial Intelligence Analyst sends an alert to the Heath Coach if a symptom worsens dramatically or if progress stagnates for too long that the Health Coach might or might not notice himself
Health Coaches have the option of sending confidential messages to another Health Coach with a question on how to proceed
The Artificial Intelligence Analyst reviews patient independently of the Health Coach, and sends recommendations to the Health Coach for him to decide. It is human rather than artificial intelligence that is forwarded to the Patient.

### VIII. Description of Some System Components That Carry Out Our Method

Main Components include the Account Setup and Intake, Foods, Mealtimes and NonFoods, Health Tracker and Symptoms, MyPlan and Patient Response, and the Notice Board. Motivation microservices are the Buddy system, the social media platform, and the long-term progress chart based on the data that the patient input. See sections following for descriptions and illustrations of many components. Sub-components include Messaging and Scheduling between patient and health coach. Components accessible to the Provider only are the Patient Background Chart, the Artificial Intelligence Analyst and the Knowledge Base.

For the 3 main user groups of the system, Patients, Providers, and Administrators, there are 3 different portal interface (See FIG. 1). Notice that different tasks and services are available to different user groups although all are described in the section following.

### IX. Description and Purpose of Some Invention Components

Account: We need a way to contact patients within and also without the system for legal purposes. Here, patients enter contact information over secure, encrypted channels. Additional confidentiality comes from their setting up in-portal names distinct from their legal names.

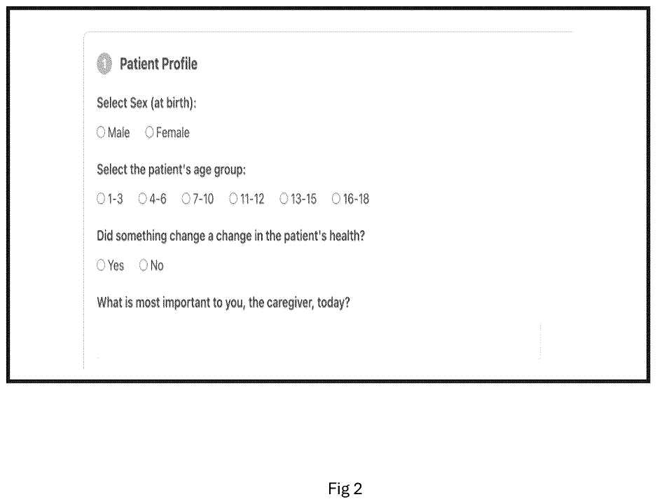

Intake: An introductory personal assessment is requested of each patient so that the system has the basis to personalize the program. (FIG. 2) We might ask questions such as ‘what are your eating habits?’ or as per Dr Nathan, ‘what is the number one problem you are dealing with? (Dr Nathan, Toxic, 2018, p. 22).

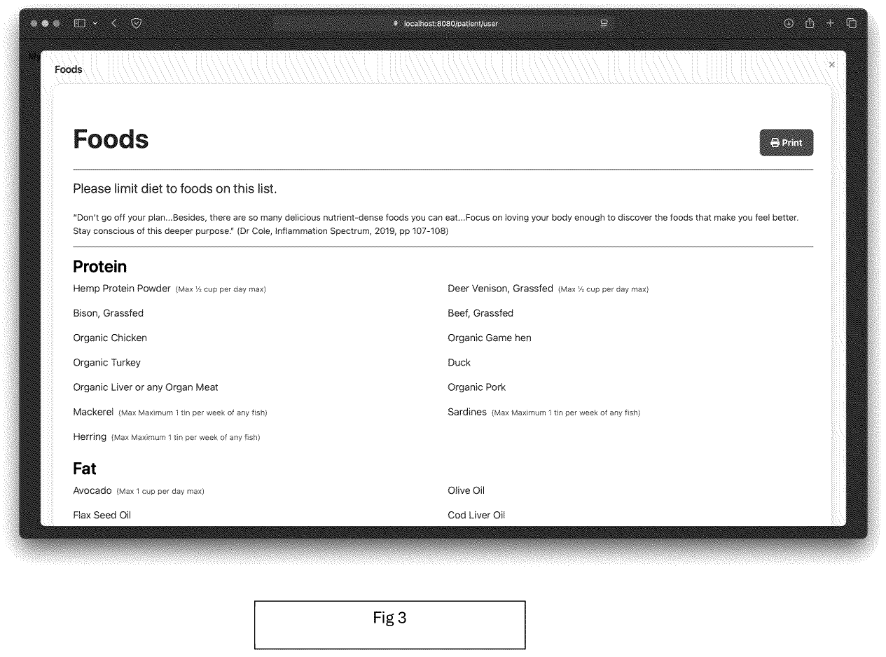

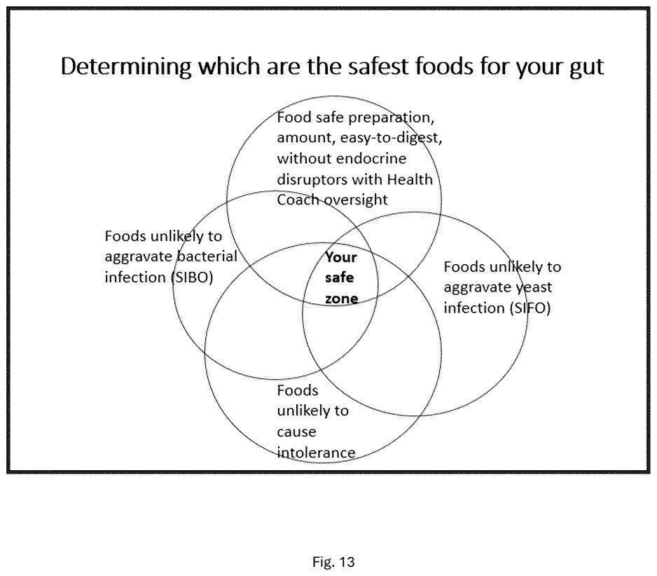

Foods: This is a macronutrient list of potential foods for you to eat, in what quantity and with what preparation (FIG. 3), in some cases, with a separate section for recipes based on the foods recommended. Foods periodically becomes a checklist, to allow the Health Coach to see what the patient is eating. Each person's Foods list is updated periodically based on that person's responses to those foods. For the reasoning behind the low carbohydrate-type foods recommended at the beginning of the program, see FIG. 13.

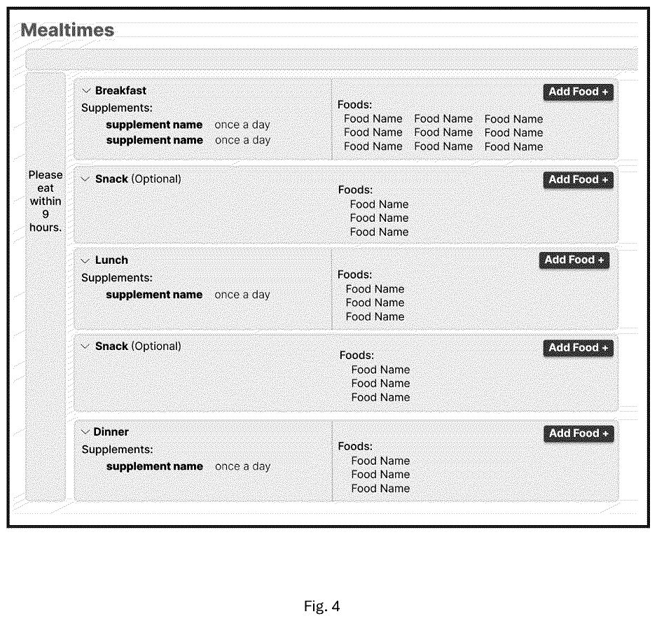

Mealtimes: Micronutrients may be found in macronutrient foods. However, micronutrients isolated in dietary supplements might be more practical than foods in some cases owing to the availability and seasonal nature of particular foods, or the quantity of food required by an individual patient. (For example, depending on the variety of the orange and its ripeness, it might take 4 oranges to supply the equivalent amount of vitamin C in a 250 mg vitamin C tablet). This microservice combines the individual's micronutrient suggestions with timing suggestions - such as eating within a smaller window of time to encourage cellular repair (autophagy, see Glossary). It is meant as a memory aide, among other things (see FIG. 4), and it becomes a data-input worksheet periodically to allow the Health Coach to comment on what the patient is choosing.

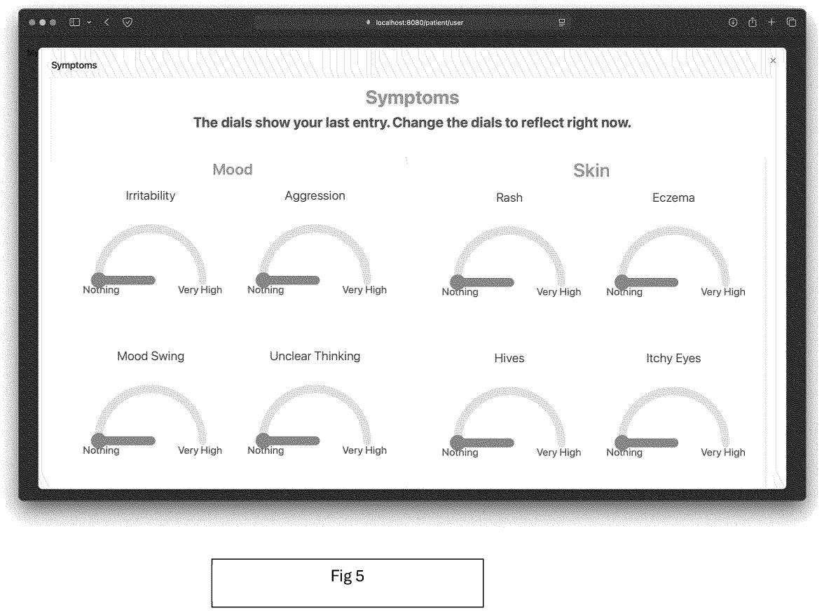

Symptoms: Patients might report how they are feeling in the Symptoms microservice so that the Health Coach can adjust their regimens. To report what's going on and at what intensity, the patient dials the intensity of each distress. (See FIG. 5) The Symptoms microservice includes write-in symptoms and a write-on basic body map so a patient can indicate an area of distress. It includes Bristol stool scale input as a measure of digestive health.

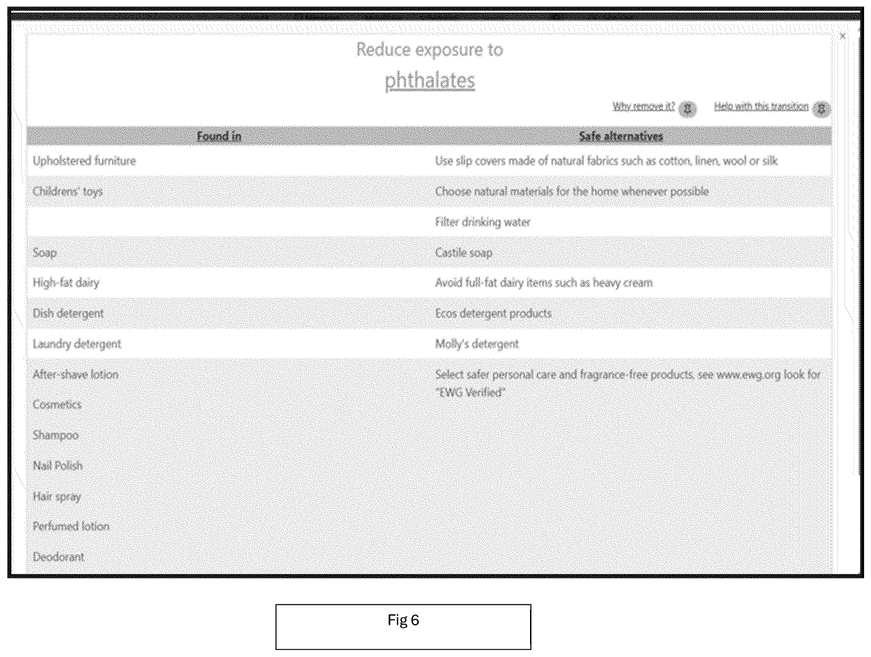

Non-Foods: This microservice outlines potential, non-obvious sources of harm in everyday products and circumstances presented with safer, healthier alternatives. In the embodiment (FIG. 6), the chemical name is listed center top screen, a list of products that might contain that chemical is the left-hand column, and a list of alternatives without that chemical is in the right-hand column. Non-Foods does not focus on widely-recognized sources of harm such as smoking or drinking alcohol, or ingesting items for which most would contact a poison control center. Instead, it concentrates on non-obvious sources of minute quantities of toxins that might cause harm when used over time.

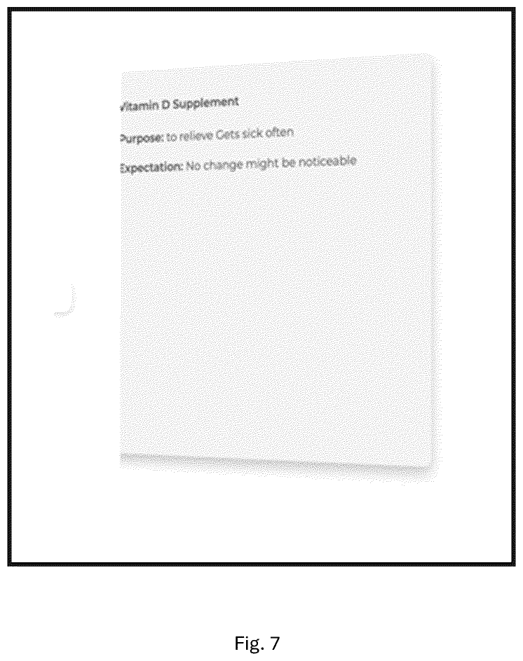

My Plan: A personalized suggestion from a Health Coach, with detail from the Knowledge Base might be sent to the Patient in a Plan “card”. Examples of types of suggestions: food preparation or supplements to improve digestion, or an herb with antimicrobial properties to lessen microbial overgrowth in the small intestine. A suggestion includes a reference to further reading in the event that the Patient wants to learn more in deciding whether to follow that suggestion. Product-related suggestions might include a link to a product for patient convenience. See embodiment with a “card” for each suggestion (FIG. 7).

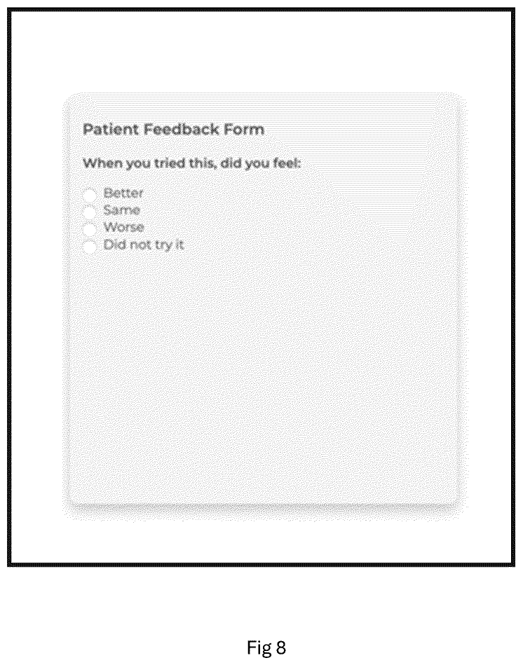

Patient Response: Each personalized suggestion might be sent separately, with a response form that accompanies the suggestion card (FIG. 8). The patient is asked to respond whether a suggestion was tried, and if so, whether or not it made a positive health difference. This Patient Response to suggestions is part of the feedback loop to personalize nutrition. For convenience, the Patient Response to a suggestion card (FIG. 8) might be on the flip side, that is the virtual back of that suggestion card (FIG. 7).

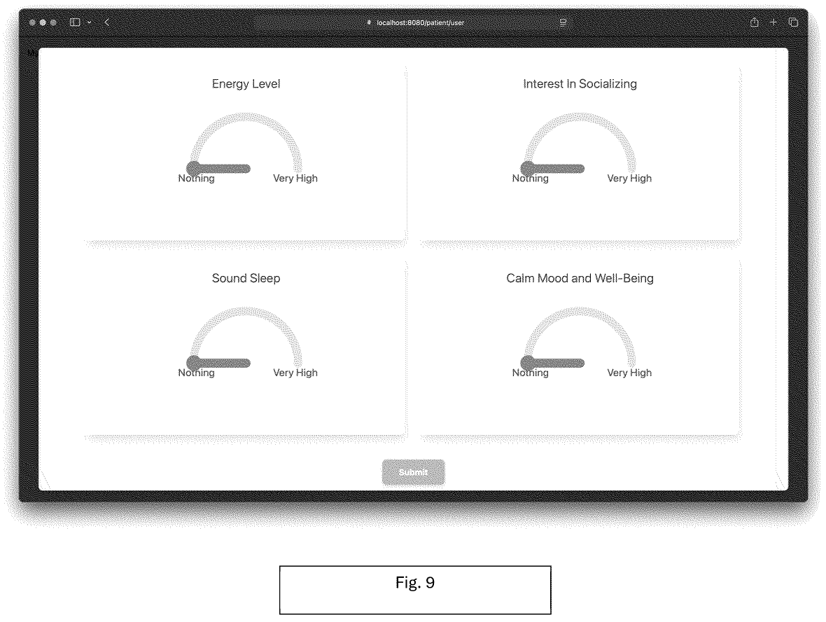

Health Tracker: The purpose is to give an index of general health and well-being using a few key factors such as energy level. The patient moves the dial based on the level of—for example energy—in comparison to the level experienced during the month prior (See FIG. 9).

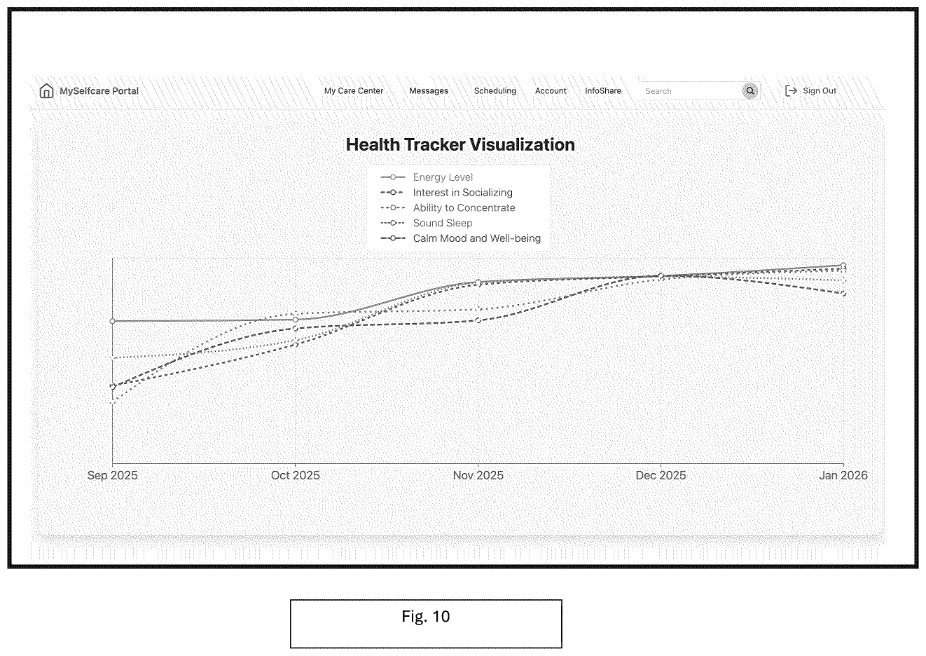

Health Progress Visualization Chart: This chart represents the System's visualization of the Patient's Health Tracker data. Healing the gut might take a long time because the intestines are long, having a lot of surface area. For that reason, we use a sensitive scale that can show healing even in small increments. We hope that visualizing the patient's own progress over the long term might inspire some to continue with the program. (See FIG. 10)

Notice Board+Messages: The read-only Notice Board allows gentle directions to Patients that might concern the program, or motivational statements from a Provider, or System messages about temporary website inactivity, etc. The Administrator may post Notices about the program or may alert the users in the event of an emergency, such as a service outage. The Message Center, on the other hand, sets up secure communication within the portals between a Patient and Provider, between two Patient Buddies, or between a Patient and the Administrator.

Scheduling: Patients who wish the option of secure, encrypted meetings with the Food Therapist or Health Coach can schedule a meeting time based on mutual availability. At the appointed time, the system establishes a secure, encrypted channel between Patient and Health Coach appropriate to the desired voice or video communication. Such meetings in most cases will be optional rather than required by the program.

Buddy System: This might pair adult patients or to pair patient-caregivers. The basis of the match could be, for example, if two people are comfortable in a particular language, or example, or if they are caregivers of same-age patients. If both sides independently accept the proposed anonymous buddy, then a confidential channel is provided for encrypted communications between buddies. In future, we might invite graduates of the program to return as buddies to encourage those who are just starting.

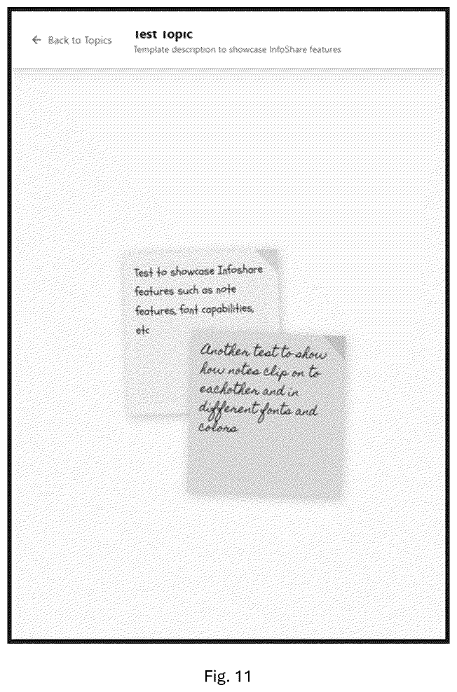

Social media: Whiteboards typically are used for business purposes so that different people can be assigned or contribute to a goal or topic. Instead, we use a whiteboard as a type of social media platform, so each person can to join the in-portal community and have their say on a variety of topics relevant to the program. For instance, a topic might inspire those following our program (does the breakfast meal want different foods from lunch and dinner?) or to look beyond our program whether they are travelling, managing holidays or pets, in a school setting, or assisting the elderly. We encourage contributors to use an in-portal alias for security reasons. The topics will be changed by the Administrator periodically. We designed InfoShare to be pleasant to look at and fun to do. (See FIG. 11) Choose your handwriting style and note color, write your thought, and position your message anywhere on the board to stand alone or respond to another contributor's message.

Patient Background Chart [available to Health Coaches Only] and My Care Center [available to the Patient only]: These include much of the same information, but that information is presented and indexed for different purposes. For the Health Coach, the purpose of Patient Chart is to give an overview about each patient and draw attention to areas in which the patient might feel unwell or need assistance. While for the Patient,

the individual's own information and Health Coach notes can be searched by date or keyword.

Artificial Intelligence Analyst [available to Health Coaches only]: The purpose is to add reliability and versatility to the program, support the Health Coaches and improve Patient health outcomes. The Artificial Intelligence Analyst is supported by the Knowledge Base (which is itself updated by Health Care staff and Administrators continually). The AI Analyst might draw a conclusion and make a suggestion that the Provider did not consider, acting like a team supporting the Provider. All AI Analyst suggestions, however, are considered by the Provider and only what the Provider decides will be sent to the Patient.

Long-term when the System serves thousands, the AI Analyst will rely on machine learning from anonymous data. Presently it is rules based. Examples of AI Analyst tasks:
Point to a food or food group using food journal logic that might be troubling the patient
Consult the Knowledge Base for non-pharmacological suggestions or relief for a gut dysbiosis condition
Notify the Healthcare Provider that the Patient might be ready to advance to the next stage of the gut repair protocol
Alert the Healthcare Provider as to a patient symptom that remains entrenched or worsens over a period of time
Adjust the optimal amount of a nutrient and resume foods as the patient gets better

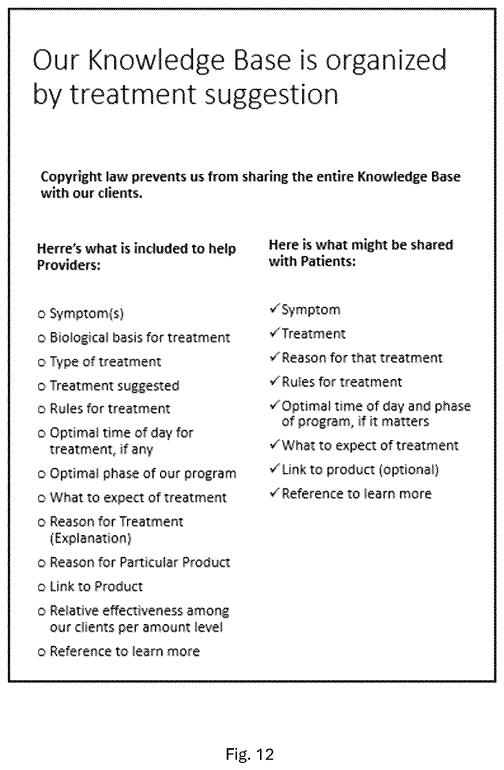

Knowledge Base [available to Health Coaches only]: The Knowledge base is a continually updated and expanding proprietary reference that was assembled in-house. As such, it cannot be open to the public for liability reasons, but must be consulted in-house only by the Health Coaches and the AI Analyst. Sources of knowledge in the Knowledge Base include references to books by medical practitioners, the experience of our Health Coaches, and updated references to the scientific and medical literature. Providers are encouraged to add their own suggestions or make updates to the Knowledge Base. FIG. 12 is a chart showing what types of information are stored for the Health Coaches and what types of information are able to be shared with Patients.

Records Management [available to the Administrator only]: Laws require maintaining documentation for Patients and Health Coaches too. The system follows Health Insurance Portability and Accountability Act and related legal requirements, and will continue to be updated as laws are changed. For example, federal and state records retention policies specify the number of years that patient records must continue to be encrypted and retained, even after the patient has graduated out of the system. This section for legal reasons is available to the Administrator only.

### X. Key Program Innovations in our Method and System

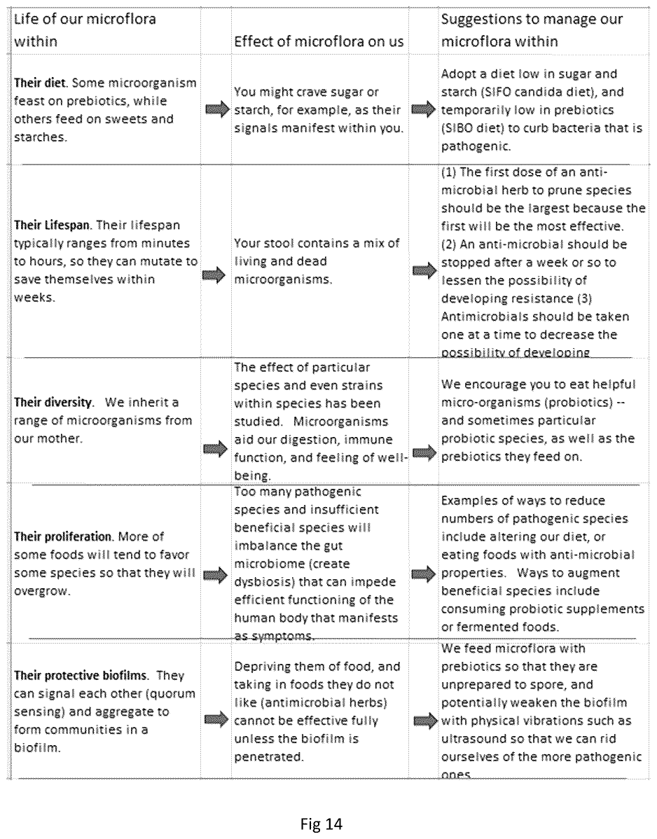

A. Beginning “safe” foods (FIG. 13)
B. Gut re-balance method (FIG. 14)
C. Personalized nutrition (FIG. 15)
D. Value-based care

### A. Beginning “Safe” Foods Suggestions

The reasoning behind the patient's starting diet is diagrammed (FIG. 13). The starting diet accounts for foods that do not aggravate the commonly-experienced Small Intestinal Bacterial Overgrowth (SIBO) (Davis, Super Gut 2022, p.7), as well as Small Intestinal Fungal Overgrowth (SIFO) which is known to co-occur often with the Small Intestinal Bacterial Overgrowth (SIBO). Therefore, our starting diet recommendations consist of those foods in amounts that are acceptable for both SIBO, small intestinal bacterial overgrowth (as expressed and SIFO, small intestinal fungal overgrowth. Safe foods for a SIBO diet are listed in Kellman, Microbiome Diet, 2014, and safe foods for a SIFO diet are listed in Perkins, Chronic Candida, 2018. But that beginning diet is a form of

low-carbohydrate diet. The beginning diet resembles a modified version of the diet called “paleo,” with meat and fish, vegetables and non-sweet fruits, and without grains or dairy.

We add the protection of the intermittent fasting—essentially a delayed breakfast—to enhance cellular repair in autophagy (Ohsumi Y. Historical landmarks of autophagy research. Cell Res. 2014 January; 24(1):9-23.). Together, this is presented in the system's Mealtimes and Foods. Foods are introduced and the list is refined soon after patient interaction with the system. Related to this is the removal of substances that aggravate digestion such as pesticides and antibiotics. This list of potential trace toxins that might interfere with bodily function is presented gradually throughout the program.

### B. Key Aspects of our Gut Re-balance Effectiveness

We need to understand the microorganisms within each of us to improve our human health. Research has demonstrated that the out-of-balance condition known as Small Intestinal Bacterial Overgrowth (SIBO) has been associated with several hundred conditions (See Dr Siebecker's list on https://www.siboinfo.com/sibo-associated-conditions.html, accessed Nov. 25, 2025). For that reason, we aim to reduce the overgrowth of SIBO as effectively as possible.

While the basic strategy for re-balance of the microorganism populations derives from Dr Bland and functional medicine (Bland, Disease Delusion, 2014), that strategy has been outlined succinctly by Dr Kellman (Kellman, Microbiome Diet, 2014). However, clinical practice (as described in Davis, Super Gut, 2022; Kellman, Microbiome Diet, 2014, and in office practice, (Dr Wilson, personal communication, 2023), is direct Patients to take multiple antimicrobials at a time, for several weeks at a time, and in minor doses so as not to stir up a severe “herx reaction” (See Glossary of this disclosure). Products that bind to and help the body remove toxins can be used to lessen the herx effects. (See Perkins, Healing Chronic Candida, 2018, p. 215-16)

But the scientific literature reveals that antimicrobial doses that begin low, and antimicrobials taken several at a time encourages the development of resistance. That is, the structures of the microbes mutate to allow them to withstand anti-microbials and make treatment less effective. Furthermore, some bacteria strive to save their lives by clumping together, living in a protective biofilm. The biofilm includes “persister” cells that do not succumb to herb treatment, and these reproduce, forming a colony. The logic for this gut microbiome re-balance strategy is outlined in FIG. 14.

In short, our program improves upon gut re-balance strategy by adjusting for the microbial life cycle. In-depth consideration of the research leads us to suggest a process (FIG. 14 column 3) that indicates greater effectiveness.

### C. Personalized Nutrition

Significance. A patient experiencing an over-active immune response might respond to even an ordinary, non-processed food with inflammation, which the patient experiences as a food intolerance. “It's crucial to identify and stop eating foods you're sensitive to, as they only fuel inflammation.” (Dr Moday, Immunotype Breakthrough, 2021 p. 224).

Method. Dieticians discern the foods causing problems with the help of a food diary or food journal. Dieticians recommend that it should include foods and dietary supplements, symptoms, and bowel movements (Jacob, Digestive Health, 2013, p153). Our system analyzes macro and micronutrients and symptoms in this way to point to foods which might be problematic. Is considering foods and timing together enough? Dr Moday explains that “tests can be really helpful [such as IgG food sensitivity test], but I want to be clear that you do not need them as proof before you start taking major steps to squelch your immune imbalances.” (Dr Moday, Immunotype Breakthrough 2021, p. 224)

Logic of implementation. It is known that frequently-eaten foods are more likely to cause intolerances. It is known also that foods with certain compounds cause certain types of distress. For example, high levels of histamine can lead to headaches, disrupted sleep, attention deficit, crying easily, abnormal fears and other distresses. (Perkins, Chronic Candida, 2018, p.445) Goods high in histamine in a food might vary based on food preparation, for example. Many types of nut are high in histamines as are fruits that are dried and cheese that is aged. But the level of histamine in a food can vary based on food preparation or ripeness (Perkins, Healing Chronic Candida, 2018, p. 347-350).

System implementation. FIG. 15 shows the key services involved in the personalized nutrition process. An upper level overview is presented as follows. Step 1 is for users to select among our macro and micro-nutrient recommendations. Step 2 is for users to record what they ate and how they felt. Step 3 is for the trained Health Coach and Artificial Intelligence Analyst to review symptoms (output) in light of foods and non-foods (input), and try to determine what input might be responsible for the output, with the Health Coach selecting recommendations for macro and micronutrients to send to the user. Step 4 is for the user to try at least some of the recommendations and report back to the system whether he felt the same or better, worse, or did not try it. Step 5 is to update that user's macro and micronutrient lists based on what was tried and alright. Any macro foods or micronutrient products that made the user feel worse are sent back fore-assessment to the Provider and the AI Analyst.

### D. Value-based Care

We build metrics into our system to demonstrate value-based care. For example, to demonstrate value to Patients for improved quality of life, we can average anonymized data from the chart visualizations that show health improvement over time. To demonstrate value for the program's subscription payer, we can point to something like relieving the burden on the patient's health ecosystem with fewer visits to external Providers on account of patient health improvement with our System.

To demonstrate value for our Health Coaches who devote additional time to some patients, we configure separate scheduling and payment outside the service that goes to the Coach directly so that additional effort for case complexity is rewarded. We might also consider as a value metric the number of patients per Provider who remain motivated to continue with the program (metric suggested by Nour, Co-Create, 2017, p 169).

### XI. Hardware, Etc.

The system of the disclosure generally comprises a processor, a network interface and a memory. According to some embodiments, the memory comprises logic (e.g., instructions) that can be executed by the processor to perform various methods. For example, the logic may include a user interface module as well as a data aggregation

and correlation software application that is configured to provide the functionalities described in greater detail herein including systems and methods of patient wellness monitoring and management.

The computer system embodied in the subject disclosure includes a processor or multiple processor(s) (e.g., a central processing unit (CPU), a graphics processing unit (GPU), or both), and a main memory and static memory, which communicate with each other via a bus. The computer system may further include a video display (e.g., a liquid crystal display (LCD)). The computer system may also include an alpha-numeric input device(s) (e.g., a keyboard), a cursor control device (e.g., a mouse), a voice recognition or biometric verification unit, a drive unit (also referred to as disk drive unit), a signal generation device (e.g., a speaker), and a network interface device. The computer system may further include a data encryption module to encrypt data.

The disk drive unit includes a computer or machine-readable medium on which one or more sets of instructions and data structures are stored embodying or using any one or more of the methodologies or functions described herein. The instructions may also reside, completely or at least partially, within the main memory and/or within the processor(s) during execution thereof by the computer system. The main memory and the processor(s) may also constitute machine-readable media.

The instructions may further be transmitted or received over a network via the network interface device using any one of several well-known transfer protocols (e.g., Hyper Text Transfer Protocol (HTTP)). While the machine-readable medium is shown in an example embodiment to be a single medium, the term “computer-readable medium” should be taken to include either a single medium or multiple media (e.g., a centralized or distributed database and/or associated caches and servers) that store one or more sets of instructions.

The term “computer-readable medium” shall also be taken to include any medium that can store, encode, or carry a set of instructions for execution by the machine and that causes the machine to perform any one or more of the methodologies of the present application, or that is capable of storing, encoding, or carrying data structures used by or associated with such a set of instructions. The term “computer-readable medium” shall accordingly be taken to include, but not be limited to, solid-state memories, optical and magnetic media, and carrier wave signals. Such media may also include, without limitation, hard disks, flash drives or external storage media, digital video disks, random access memory (RAM), read only memory (ROM), and the like. The example embodiments described herein may be implemented in an operating environment comprising software installed on a computer, in hardware, or in a combination of software and hardware.

One skilled in the art will recognize that the Internet may accessed through any internet-enabled device. Those Internet-enabled devices may include one or more processors, buses, memory devices, display devices, input/output devices, and the like. Furthermore, those skilled in the art will appreciate that Internet service may be coupled to one or more databases, servers, and the like, which may be used to implement any of the embodiments of the disclosure as described herein.

Our Patient Portal, Provider Portal and Administrator Portal, read over an Internet connection, rely on program instructions that may be loaded onto a computer, other programmable data processing apparatus, or other devices to cause a series of operational steps to be performed on a computer server, other programmable apparatus or other devices to produce a computer implemented process such that the instructions which execute on the computer or other programmable apparatus are able to implement the functions specified.

Our Portals preferably require browser Internet access to one or more computing devices, although the system could be configured as a downloadable app (that is, application), that works otherwise. The computing devices may include one or more processors, buses, memory devices, display devices, input/output devices, and the like. Furthermore, those skilled in the art may appreciate that the Internet service may be coupled to one or more databases, repositories, servers, and the like, which may be used to implement any of the embodiments of the disclosure as described herein.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel or may be performed at different times.

While various embodiments have been described above, they are presented as examples only, and not as a limitation. The descriptions are not intended to limit the scope of the present technology to the forms set forth herein. To the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described embodiments that are exemplary.

What is claimed is:

1. A computer-implemented method for updating healthcare suggestions for a patient, the method comprising:
receiving, via a profile interface of an intake user interface module executing on at least one processor, a plurality of profile inputs from the patient;
presenting, via a food data user interface module executing on the at least one processor, food data comprising a plurality of food items, most food items having an associated consumption indicator;
presenting, via a meal timing module executing on the at least one processor, recommended micronutrient recommendations;
presenting, via a toxin information module executing on the at least one processor, information regarding potential toxins in household products;
receiving data on patient choices from the patient macronutrient, micronutrient, toxins, and
generating, using an artificial intelligence system executing on the at least one processor, treatment suggestions based on the plurality of profile inputs and indicators received from the food data user interface module, the meal timing module, and the toxin information module;
transmitting artificial intelligence-generated treatment suggestions to a healthcare professional for decision review and
allowing the healthcare professional the optional of adding to or creating new treatment suggestions for the patient; and then transmitting those decided-upon treatment suggestions to the patient;
receiving, via the user interface, patient feedback regarding the healthcare professional's transmitted treatment suggestions; and when appropriate,
updating at least one of the macronutrient or micronutrient recommendation lists based on that patient feedback.

2. The method of claim 1, further comprising:
periodically updating the treatment suggestions based on patient feedback received in response to previously transmitted treatment suggestions.

3. The method of claim 1, further comprising:
correlating macronutrients and micronutrients consumed by the patient with patient-reported symptoms over time; and
modifying food recommendations by reducing or removing foods, or adding foods as associated with the patient-reported symptoms.

4. The method of claim 1, wherein generating the treatment suggestions comprises:
retrieving, by the artificial intelligence system, information related to treatment from medical or scientific research publications; and
presenting that treatment information to the healthcare professional, allowing the professional to select what to sent to the patient.

5. The method of claim 1, further
providing an optional empathy module executing on the at least one processor, an encrypted communication channel that enables the patient to exchange empathy-related communications with another patient; and
providing an optional community module executing on the at least one processor, a sort of confidential community forum enabling the patient to interact with others going through the same program.

6. The method of claim 1, wherein references to deeper educational content is offered to those users who wish to learn more.

7. The method of claim 1, further comprising the
personalizing a task interface to display only tasks relevant to that individual's current treatment stage.

8. The method of claim 1, wherein receiving the patient feedback comprises:
receiving a description of a discomfort experienced by the patient;
receiving a location indicator in some cases to indicate where the discomfort is experienced;
receiving a severity indicator indicating the discomfort's severity; and
comparing the severity indicator to a previously reported severity indicator from a prior feedback cycle.

9. The method of claim 1, further comprising a:
periodical update of macronutrient recommendations and micronutrient recommendations throughout the duration of the wellness program based on patient feedback.

10. The method of claim 1, wherein presenting the recommended meal times comprises:
determining and at an optimal time presenting treatment based on the patient's phase of healing; and
presenting the optimal time of day to carry out the healthcare provider's treatment suggestions to improve absorption, and the presenting time to refrain from eating.

11. The method of claim 1, wherein presenting information regarding potential toxins comprises:
presenting categories of products that might include trace amounts of a toxin,
offering safer alternatives to those products; and
presenting toxin categories gradually over time.

12. The method of claim 1, further comprising
The periodic receiving, via the user interface, of the status a plurality of health goals from the patient;
and displaying a graphical visualization of the patient progress toward the plurality of health goals.

13. The method of claim 1, further
collecting anonymous statistical data regarding patient responses;
analyzing that anonymous statistical data to generate metrics comprising at least one of: treatment effectiveness, user interface usability, or the healthcare professional's success rate; and
modifying the method based on these metrics.
